(19) United States
(12) Patent Application Publication
(10) Pub. No.: US 2016/0120497 A1
(43) Pub. Date: May 5, 2016

(54) RADIOGRAPHIC IMAGING APPARATUS AND METHOD FOR CONTROLLING THE SAME

(71) Applicant: SAMSUNG ELECTRONICS CO., LTD., Suwon-si (KR)

(72) Inventors: Desai NASIR, Suwon-si (KR); Abhinav MEHROTRA, Sitapur (IN)

(73) Assignee: SAMSUNG ELECTRONICS CO., LTD., Suwon-si (KR)

(21) Appl. No.: 14/706,078

(22) Filed: May 7, 2015

(30) Foreign Application Priority Data
Oct. 31, 2014 (KR) 10-2014-0149724

(51) Int. Cl. A61B 6/00; G06T 11/60

(52) U.S. Cl. CPC A61B 6/56; G06T 11/60; A61B 6/54

(57) ABSTRACT

A radiographic imaging apparatus includes a radiation scanner; and a workstation configured to control the radiation scanner. The workstation is configured to output analyzed data for a functional error of at least one of the radiation scanner and the workstation in graphics, and output analyzed data for an item of the functional error in response to an input of a user.

### CROSS-REFERENCE TO RELATED APPLICATION

This application claims priority from Korean patent application No. 10-2014-0149724, filed on Oct. 31, 2014, in the Korean Intellectual Property Office, the disclosure of which is incorporated herein in its entirety by reference.

### TECHNICAL FIELD

Apparatuses and methods consistent with exemplary embodiments relate to a radiographic imaging apparatus for scanning an object, and a method for controlling the radiographic imaging apparatus.

### BACKGROUND

Radiographic imaging apparatuses image an internal region of an object using radiation, such as X-ray, that is absorbed or transmitted depending on the property of a substance included in the object. The radiographic imaging apparatus may provide an image of the internal region of the object to the user by using radiation transmitted through the object or generated from the internal region of the object and generating a radiographic image based on electric signals output based on the received radiation.

The radiographic imaging apparatus is widely used in many different industrial fields because the radiographic imaging apparatus allows a user to identify the internal structure of an object. For example, the radiographic imaging apparatus may be used in hospitals to detect lesions in human bodies or in factories to detect the internal structure of an object or a part. The radiographic imaging apparatus may also be used for border control at airports to check luggage.

Examples of the radiographic imaging apparatus may include a digital radiography (DR) device, a computed tomography (CT) device, a full field digital mammography (FFDM), or the like.

### SUMMARY

One or more exemplary embodiments provide a radiographic imaging apparatus and a method for controlling the same, which analyze functional errors of the radiographic imaging apparatus in real time, and provide the analyzed data in a graphical form to a user.

One or more exemplary embodiments also provide a radiographic imaging apparatus and a method for controlling the same, which analyze functional errors of the radiographic imaging apparatus in real time or send the functional errors to a server, and provide the analyzed data in a graphical form to the user.

In accordance with an aspect of an exemplary embodiment, a radiographic imaging apparatus includes a radiation scanner; and a workstation for controlling the radiation scanner, wherein the workstation outputs analyzed data for functional errors of the radiation scanner or the workstation in graphics, and outputs analyzed data for a detailed item of the functional error in response to an input of a user.

The workstation may output the analyzed data for the detailed item of the functional error in graphics.

The workstation may include a storage unit in which the analyzed data for the functional errors and the analyzed data for the detailed item are classified and stored, and the workstation may generate the analyzed data for the functional errors and the analyzed data for the detailed item based on types of components and check items included in the radiation scanner or the workstation, and store the analyzed data for the functional errors and the analyzed data for the detailed item in the storage unit.

The workstation may output the analyzed data for the functional error in the form of a chart or a graph.

The workstation may output the analyzed data for the functional error in time series.

The workstation may detect the functional error in real time.

The workstation may send data regarding operation of the radiation scanner or the workstation to a server, and control operation of the radiation scanner or the workstation based on an error correction signal received from the user or the server.

The workstation may receive an error correction signal from the user and accordingly control operation of the radiation scanner or the workstation.

In accordance with an aspect of an exemplary embodiment, a radiographic imaging apparatus includes: a controller for detecting functional errors of a radiation scanner and generating analyzed data for the functional errors and analyzed data for a detailed item of the functional errors; an output unit for outputting the analyzed data for the functional errors in graphics; and an input unit for receiving a detailed item for the functional errors, wherein the output unit outputs the analyzed data for the received detailed item.

The input unit may receive an error correction signal from a user and the controller may control operation of the radiation scanner based on the error correction signal.

The radiographic imaging apparatus may further include: a storage unit in which the analyzed data for the functional errors and the analyzed data for the detailed item of the functional errors are classified and stored, wherein the controller generates the analyzed data for the functional errors and the analyzed data for the detailed item of the functional errors based on types of components and check items included in the radiation scanner, and stores the analyzed data for the functional errors and the analyzed data for the detailed item of the functional errors in the storage unit.

The output unit may output the analyzed data for the functional errors in the form of a chart or a graph.

The output unit may output the analyzed data for the functional errors in time series.

The controller may detect the functional error in real time.

The analyzed data for the detailed item of the functional errors may include analyzed data for at least one of mechanical errors of the radiation scanner, input errors due to erroneous inputs of a user, and transmission errors due to transmission or reception of erroneous information.

In accordance with an aspect of an exemplary embodiment, a method for controlling a radiographic imaging apparatus is provided. The method includes generating analyzed data for functional errors of a radiation scanner or a workstation that controls the radiation scanner; outputting the analyzed data for the functional errors in graphics; receiving a detailed item of the functional errors; generating analyzed data for the detailed item of the functional errors; and outputting the analyzed data for the detailed item of the functional errors.

The method may further include storing the analyzed data for the functional errors and the analyzed data for the detailed item of the functional errors in a storage unit.

Generating analyzed data for functional errors may include generating the analyzed data for functional errors based on types of components and check items included in the radiation scanner or the workstation.

Outputting the analyzed data for the functional errors may include outputting the analyzed data for the functional errors in a chart or a graph.

Outputting the analyzed data for the functional errors may include outputting the analyzed data for the functional errors in time series.

The method may further include: receiving an error correction signal from a user; and controlling operation of the radiation scanner or the workstation based on the error correction signal.

### BRIEF DESCRIPTION OF THE DRAWINGS

The above and/or other aspects will become more apparent by describing certain exemplary embodiments with reference to the accompanying drawings, in which:

FIG. 1 is a block diagram of a radiographic imaging apparatus, according to an exemplary embodiment;

FIG. 2 is a view of a computed tomography (CT) apparatus, according to an exemplary embodiment;

FIG. 3 illustrates a CT scanner, according to an exemplary embodiment;

FIGS. 4A, 4B, and 4C are block diagrams of a CT apparatus, according to exemplary embodiments;

FIG. 5 illustrates a radiation tube, according to an exemplary embodiment;

FIG. 6 illustrates a radiation detector and a collimator, according to an exemplary embodiment;

FIG. 7 illustrates radiation scanning with a CT apparatus, according to an exemplary embodiment;

FIG. 8 is a diagram for describing respective components of a central processing unit (CPU) of a CT apparatus according to an exemplary embodiment;

FIG. 9 is a diagram for describing a control process of a CT apparatus according to an exemplary embodiment;

FIGS. 10, 11, 12A, 12B, 12C, and 13 illustrate user interfaces output by an output unit of a CT apparatus according to exemplary embodiments; and

FIG. 14 is a flowchart illustrating a method for controlling a CT apparatus, according to an exemplary embodiment.

### DETAILED DESCRIPTION

Reference will now be made in detail to exemplary embodiments with reference to the accompanying drawings, wherein like reference numerals refer to like elements throughout. In this regard, the exemplary embodiments may have different forms and should not be construed as being limited to the descriptions set forth herein. Accordingly, the exemplary embodiments are merely described below, by referring to the figures, to explain aspects of the present description

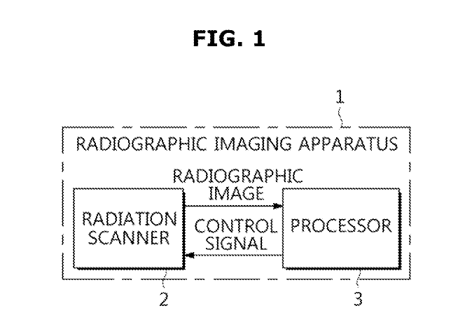

FIG. 1 is a block diagram of a radiographic imaging apparatus, according to an exemplary embodiment. Referring to FIG. 1, the radiographic imaging apparatus 1 may include a radiation scanner 2 for obtaining a radiographic image of an object, and a processor 3 as a controller for controlling an operation of the radiation scanner 2.

The radiographic imaging apparatus 1 may include various radiographic devices that may scan an object with radiation. For example, the radiographic imaging apparatus 1 may include a digital radiography (DR) device, a mammography, a computed tomography (CT) device, or the like. In addition, the radiographic imaging apparatus 1 may include any apparatus that may obtain an image of an internal region of an object with radiation. The radiographic imaging apparatus 1 may refer to a physical entity that is capable of obtaining a radiographic image, or a combination of a plurality of entities that are connected to each other via a communication network to obtain a radiographic image in association with each other.

The object may be a living entity, such as a human body or an animal, or a nonliving entity, such as a component, luggage, or the like. The object may also be a phantom. The object may be all or part of a particular object. For example, the object may be a part of a human body, e.g., a limb or an organ.

The radiation scanner 2 may perform a function to obtain a radiographic image of the object with radiation. The radiation scanner 2 may obtain the radiographic image by transmitting radiation to the object, receiving radiation transmitted through the object, and converting the received radiation to an electrical signal. The radiographic image may be raw data. The radiation scanner 2 may include a radiation source and a detector for obtaining the radiographic image. The radiation source may include a radiation tube, which may be controlled by an applied tube current and tube voltage. The radiographic image obtained by the radiation scanner 2 may be sent to the processor 3.

The processor 3 may generate a control signal to control an operation of the radiation scanner 2 based on the radiographic image sent from the radiation scanner 2, and send the control signal to the radiation scanner 2. The processor 3 may be implemented with one or more integrated chips included in the radiographic imaging apparatus 1. The processor 3 may also be implemented with one or more integrated chips included in a workstation implemented, as for example, an external computer.

While the processor 3 generates a control signal based on a radiographic image sent from the radiation scanner 2 of the radiographic imaging apparatus 1, exemplary embodiments are not limited thereto. In an exemplary embodiment, the processor 3 may receive a radiographic image obtained by an imaging device other than the radiographic imaging apparatus 1 and generate a control signal to control the radiation scanner 2. The radiographic imaging apparatus 1 and the other imaging device may be homogeneous imaging devices, or heterogeneous imaging devices. In addition to the DR device, mammography device, or CT device, the other imaging device may include a position emission tomography (PET) device and/or a single photon emission computed tomography (SPECT) device.

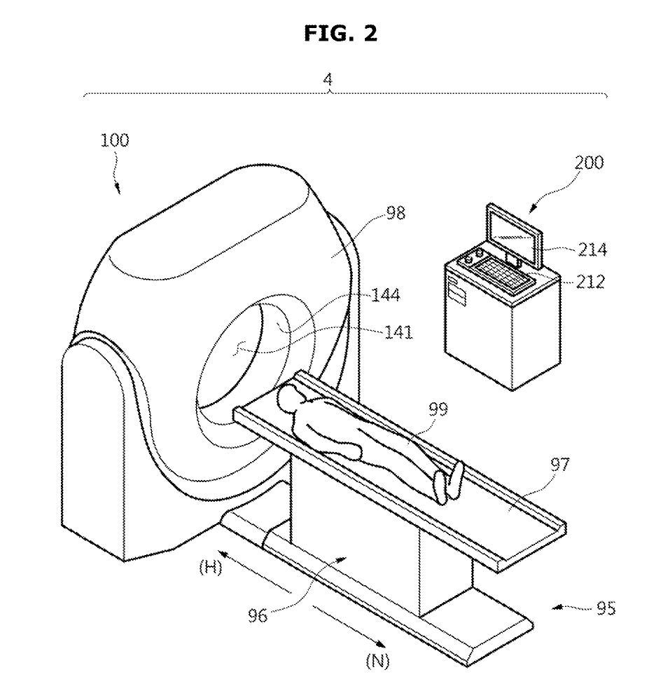

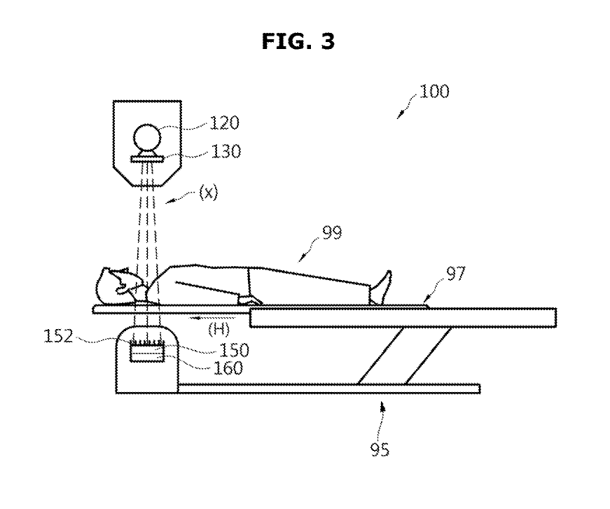

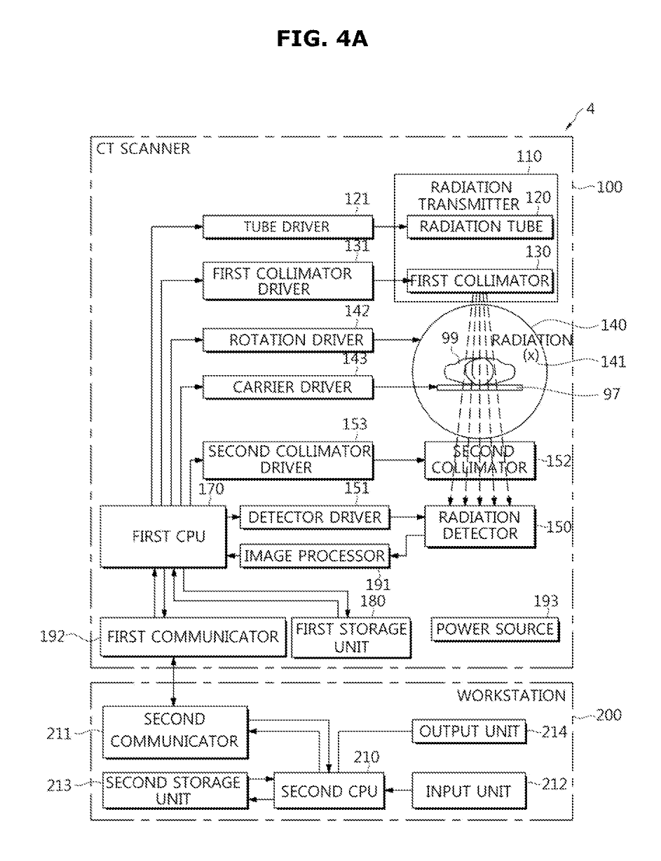

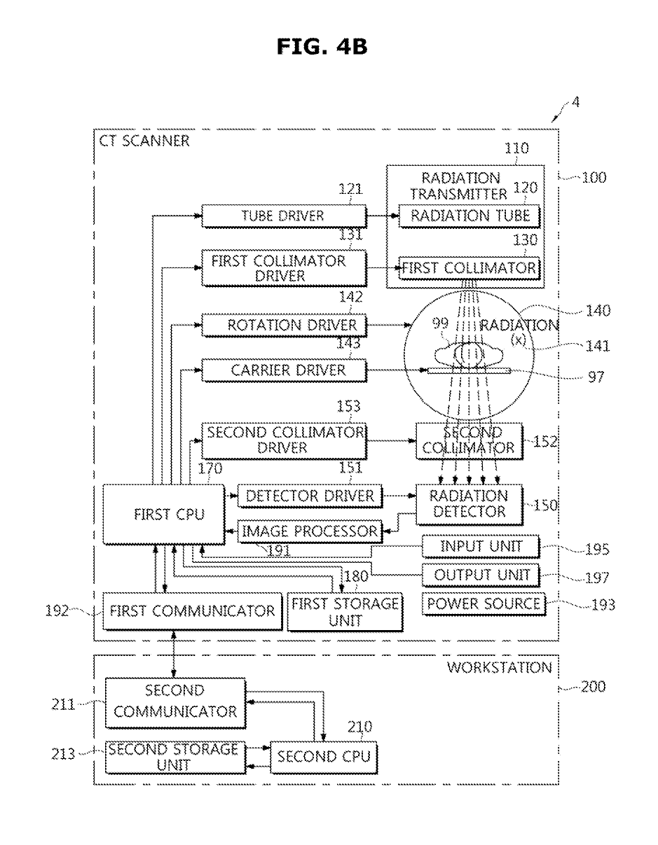

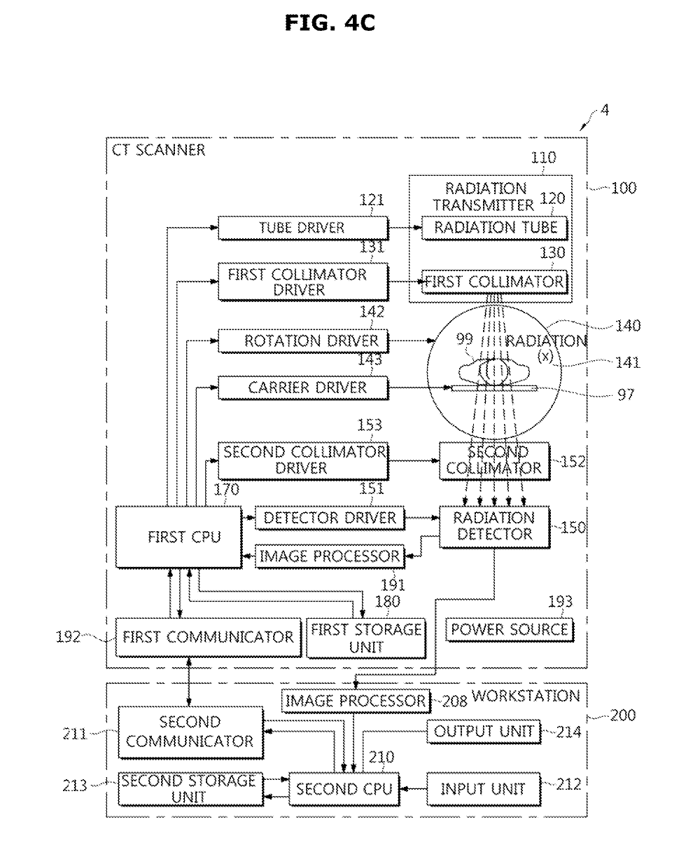

A CT apparatus will now be described with reference to FIGS. 2 to 13 as an example of the radiographic imaging apparatus 1. FIG. 2 is a view of a CT apparatus, according to an exemplary embodiment, and FIG. 3 illustrates a CT scanner, according to an exemplary embodiment. FIGS. 4A, 4B, and 4C are block diagrams of a CT apparatus, according to exemplary embodiments. Referring to FIGS. 2 to 4C, a CT apparatus 4 may include a CT scanner 100 for scanning an object, and a workstation 200 for controlling the CT scanner 100. The CT scanner 100 and the workstation 200 may be connected via a wired or wireless communication network. As shown in FIG. 4B, an input unit 195 and an output unit 197 may be included in the CT scanner 100. Alternatively, as shown in FIG. 4C, an input unit 212 and the output unit 214 may be included in the workstation 200 instead of the CT scanner 100.

As shown in FIGS. 2 and 3, the CT scanner 100 may include an exterior housing 98 that contains various components for CT scanning. A bore 141 shaped as a circle or the like may be formed in a portion in the exterior housing 98, e.g., in a center portion of the exterior housing 98. The exterior housing 98 may contain a gantry 140 capable of rotating in at least one direction. The gantry 140 may be installed along an inner circumference 144 of the bore 141. On the gantry 140, a radiation transmitter 110 and a radiation detector 150 may be installed. While the gantry 140 is rotated, the radiation transmitter 110 and the radiation detector 150 may also be rotated together.

The CT scanner 100 may include a carrier 95 for moving an object 99 in and out of the bore 141. The carrier 95 may include a cradle 97 on which the object 99 is placed, and a support 96 for supporting the cradle 97. The cradle 97 may be moved at a certain speed in a direction H toward the inside of the bore 141 of the exterior housing 98 by an operation of a carrier driver 143, such as a motor or an actuator. The moving speed of the cradle 97 may be fixed or variable. The carrier driver 143 may be included inside the support 96. Wheels or rails may be provided to the cradle 97 or the support 96 such that the cradle 97 may be moved by the operation of the carrier driver 143. In response to the movement of the cradle 97, the object 99 placed on the cradle 97 may be moved to the inside of the bore 141. After scanning is finished, the cradle 97 may be moved in a direction N which is opposite to the direction H, to transfer the object 99 out of the bore 141.

Referring to FIGS. 3 and 4, the CT scanner 100 may include the radiation transmitter 110 for transmitting radiation within the bore 141, the radiation detector 150, a second collimator 152, a tube driver 121, a first collimator driver 131, a rotation driver 142, the carrier driver 143, a detector driver 151, and a second collimator driver 153 for driving corresponding respective components, a first central processing unit (CPU) 170, a first storage unit 180, an image processor 191, a first communicator 192, a power source 180, and the like, in addition to the carrier 95 that includes the bore 141, the gantry 140, and the cradle 97. Some of the aforementioned components may be omitted, or included in the workstation 200 as shown in FIG. 4C in some exemplary embodiments.

The radiation transmitter 110 may include a radiation tube 120 for generating and transmitting radiation and a first collimator 130 for guiding the transmitted radiation.

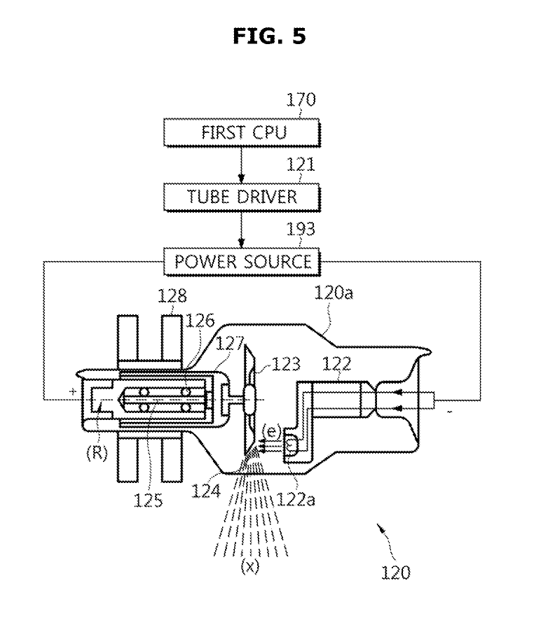

FIG. 5 illustrates a radiation tube, according to an exemplary embodiment. Referring to FIG. 5, the radiation tube 120 may be electrically connected to an external power source 193. The external power source 193 may apply a certain voltage or current to the radiation tube 120 under control of the first CPU 170 and the tube driver 121. When the certain voltage and current is applied to the radiation tube 120, the radiation tube 120 may generate radiation having a certain intensity based on the applied voltage or current. A potential difference between a cathode filament 122a and an anode 123 of the radiation tube 121 is called a tube potential, and a current generated from electrons (e) colliding against the anode 123 is called a tube current. When the tube potential increases, the velocity of the electrons (e) increases and thus the energy of the generated radiation increases. When the tube current increases, the amount of radiation may increase. Accordingly, controlling the voltage and current applied from the power source 193 may control the energy spectrum and the amount of radiation to be transmitted.

Referring to FIG. 5, the radiation tube 120 may include a capsule 120a, a cathode 122, and the anode 123. The capsule 120a may contain various components needed to generate radiation, such as the cathode 122 and the anode 123, and securely fix the components. The capsule 120a may also shield electrons (e) that are generated from the cathode 122 and moving toward the anode 123 from leaking from the capsule 120a. The capsule 120a may have a high vacuum degree of about 10⁻⁷ mmHg. The capsule 120a may include a light silicic acid glass. An electron beam (e) may be emitted from the cathode 122 toward the anode 123. The filament 122a where electrons (e) are concentrated may be arranged at an end portion of the cathode 122, and the filament 122a may discharge the electrons (e) concentrated at the filament 122a to the inside of the capsule 120a when heated by the applied tube voltage. The electrons (e) discharged from the filament 122a may be accelerated within the capsule 120a and move toward the anode 123. Energy of the electrons (e) discharged to the inside of the capsule 120a may be determined based on the tube voltage. The filament 122a of the cathode 122 may include a metal, such as tungsten W. In an exemplary embodiment, a carbon nano tube instead of the filament 122a may be provided at the cathode 122. A certain amount of radiation may be generated at the anode 123. A target plane 124 on which electrons (e) collide may be provided at the anode 126. On the target plane 124, radiation (x) having energy that corresponds to the applied tube voltage is generated due to sharp deceleration of the electrons (e) after colliding against the target plane 124. Since the target plane 124 is cut in a direction as shown in FIG. 5, the radiation (x) generated from the target plane 124 may be mainly transmitted toward a certain direction. The anode 123 may include a metal, such as copper Cu, and the target plane 124 may include a metal, such as tungsten W, chrome Cr, iron Fe, nickel Ni, or the like.

In an exemplary embodiment, the anode 123 may be a rotational anode shaped like a circular disk, as shown in FIG. 5. End parts of the rotational anode 123 may be cut at a certain angle, and the target plane 124 may be provided on the cut area of the end parts of the rotational anode 123. The rotational anode 123 may be rotated around a certain axis R at a certain speed. To rotate the rotational anode 123, a stator 128 for generating a rotating magnetic field, a rotator 127 rotated according to the rotating magnetic field generated by the stator 128, a bearing 126 rotated with the rotation of the rotator 127, and a shaft member 125 for providing the rotation axis R of the rotational anode 123 may be included in the rotation tube 120. For example, the rotator 127 may be a permanent magnet. Since the rotational anode 123 may have a higher heat accumulation rate and a reduced focus area as compared to a fixed anode, the rotational anode 123 may obtain a clearer radiographic image. In another exemplary embodiment, the anode 123 may be a fixed anode that is shaped as a cylinder and has a plane cut at a certain cutting angle, onto which electron beams (e) are transmitted. In this case, the target plane 124 may be provided on the cut part of the fixed anode. In some exemplary embodiments, the radiation transmitter 110 may include a plurality of radiation tubes 120.

The first collimator 130 may filter a plurality of radiation rays transmitted from the radiation tube 120 to guide the radiation rays to be transmitted to a region in a particular direction. The first collimator 130 may include an opening through which the radiation transmitted in the particular direction passes, and collimator blades for absorbing radiation transmitted in directions other than the particular direction. The user may use the location and size of the opening of the first collimator 130, to control the direction and range of transmission of the radiation. The collimator blades of the first collimator 130 may include a substance that may absorb radiation, such as lead Pb.

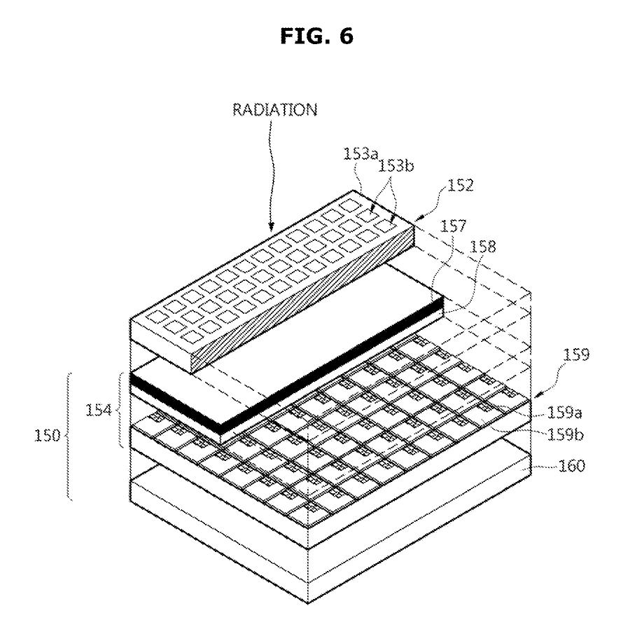

FIG. 6 illustrates a radiation detector and a second collimator, according to an exemplary embodiment. Radiation (x) transmitted from the radiation transmitter 110 may be transmitted to the object 99 inside the bore 141, pass through the object 99 to the second collimator 152, and arrive at the radiation detector 150 through the second collimator 152.

The second collimator 152 may allow only radiation that proceeds in a certain direction to arrive at a detection panel 154 of the radiation detector 150 by absorbing radiation scattered while passing through the object 99. The second collimator 152 may include a plurality of partitions 153a that block radiation and transmission apertures 153b that permit radiation to pass. The partitions 153a including a substance, e.g., lead Pb, may absorb scattered or refracted radiation, and the transmission apertures 153b may transmit non-scattered or non-refracted radiation.

The radiation detector 150 may receive radiation, convert the radiation into corresponding electric signals, and output the electric signals. In some exemplary embodiments, the radiation detector 150 may directly convert radiation into electric signals (direct scheme), or may generate visible rays corresponding to the radiation and convert the visible rays into electric signals (indirect scheme). In the case where the radiation detector 150 converts radiation into electric signals according to the direct scheme, the radiation detector 150 may include a first electrode 157 having a first surface on which radiation is incident, a semiconductor layer 158 mounted on a second surface of the first electrode 157, on which radiation is not incident, the detection panel 154 including a flat plate 159 mounted on the semiconductor layer 158, and a substrate 160 mounted on a surface of the detection panel 154. On the flat plate 159 mounted on the semiconductor layer 158, second (pixel) electrodes 159a and thin-film transistors 159b arranged in one or more columns are mounted. The first electrode 157 may have a positive (+) or negative (−) polarity, and the second electrode 159a may have a polarity opposite to the polarity of the first electrode 157. A bias voltage may be applied across the first electrode 157 and the second electrode 159a. The semiconductor layer 158 may generate pairs of charges and holes depending on whether radiation is incident on the semiconductor layer 158 or absorbed by the second collimator 152, and the pairs of charges and holes may be moved toward the first electrode 157 or the second electrode 159a depending on the polarities of the first electrode 157 and the second electrode 159a. The second electrode 159a may output an electric signal upon reception of holes or negative charges provided from the semiconductor layer 158. The thin-film transistor 159b may read out the electric signal provided from the corresponding second electrode 159a. In this case, the second electrode 159a and the thin-film transistor 159b that correspond to each other may be integrated in a single complementary metal-oxide semiconductor (CMOS) chip. In the case where the radiation detector 150 converts radiation into electric signals according to the indirect scheme, a phosphor screen that outputs visible rays based on the received radiation is arranged between the second collimator 152 and the radiation detection panel 154, and photo diodes may be arranged on the flat plate 159 instead of the second electrodes 159a to convert the visible rays to electric signals. The radiation detection panel 154 may include a scintillator for outputting a certain amount of visible photons depending on radiation and photo diodes for detecting the visible photons. The radiation detector 150 may be a photon counting detector (PCD) in an exemplary embodiment. The substrate 160 may be attached to the surface of the radiation detection panel 150 for controlling various functions of the radiation detection panel 150 or for storing electric signals output from the radiation detection panel 154.

The electric signal obtained by the radiation detector 150 may be provided to the image processor 191. The image processor 191 may generate a radiographic image on which the user may observe an internal structure of the object 99, based on the obtained electric signals, and may perform further image processing when needed. The image processor 191 may be implemented by a graphic processing unit (GPU). The GPU may include an integrated chip, such as a graphic chip. Different operations and functions of the image processor 191 may be performed by the first CPU 170 or a second CPU 210 of the workstation 200. In the latter case, the image processor 191 may be omitted from the CT scanner 100 and an image processor 208 may be instead provided in the workstation 200. The generated radiographic image may be provided to the first CPU 170 or the first storage unit 180. The radiographic image may also be provided to the workstation 200 through the first communicator 192 and a second communicator 211.

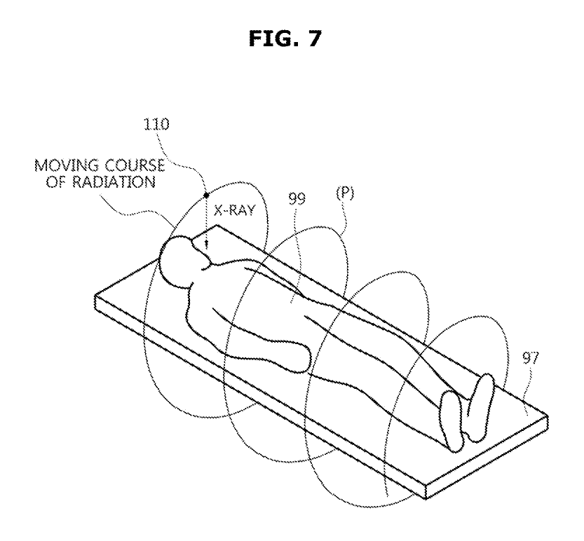

FIG. 7 illustrates radiation scanning with a CT apparatus, according to an exemplary embodiment. The radiation transmitter 110 and the radiation detector 150 may repeatedly capture radiographic images of the object 99 while being rotated by the gantry 140. As described above, since the object 99 moves toward the inside of the bore 141 by the carrier 95 at a constant speed, the object 99 is scanned while the radiation transmitter 110 and the radiation detector 150 are spirally rotated around the object 99. Consequently, scanning of the entire object 99 may be achieved. During the scanning, the object 99 may be moved at a particular speed in a region. If the particular speed in the region is slower than a moving speed of the object 99 in another region, the number of rotations of the gantry 140 in the region increases compared to that in the other region. In other words, the moving speed of the object is inverse proportional to the number of rotations of the gantry 140.

The CT scanner 100 may include the first CPU 170 for controlling respective components of the CT scanner 100. The first CPU 170 may control an operation, such as radiation scanning and image processing, of the CT scanner 100 by generating a control command according to a pre-stored setting or a selection of the user and sending the control command to the radiation transmitter 110, the second collimator 152, the radiation detector 150, the image processor 191, the gantry 140 or the carrier driver 143, and the like. When needed, the first CPU 170 may send the control command for the tube driver 121, the first collimator driver 131, the rotation driver 142, the carrier driver 143, the detector driver 151, and the second collimator driver 153 to control operations of respective corresponding components. The first CPU 170 may send the control command for the tube driver 121, the first collimator driver 131, the rotation driver 142, the carrier driver 143, the detector driver 151, and the second collimator driver 153 to control operations of the respective corresponding components. The first CPU 170 may perform functions of the processor 3 discussed in the exemplary embodiment of FIG. 1. The first CPU 170 may be implemented with one or more integrated chips, which are capable of performing functions of computing and/or processing, and may be mounted on e.g., a printed circuit board.

The tube driver 121 may apply a tube voltage and a tube current to the radiation tube 120 by turning on or off a switch connected to the radiation tube 120 according to the control command provided from the first CPU 170. The first collimator driver 131 may operate the first collimator 130 by expanding or reducing the aperture of the first collimator 130 according to the control command provided from the first CPU 170. The rotation driver 142 may rotate the gantry 140 according to the control command provided from the first CPU 170. In response to the rotation of the gantry 140, the radiation transmitter 110, the first collimator 130, the second collimator 152, and the radiation detector 150 may be rotated together. The carrier driver 143 may operate to move the cradle 97 in the direction H toward the inside of the bore 141 of the exterior housing 98, according to the control command of the first CPU 170. As discussed above, the carrier driver 143 may include a motor or an actuator. The second collimator driver 153 may operate the second collimator 152 according to the control command of the first CPU 170, and in this case, the operation of the second collimator 152 may correspond to, for example, location movement in a vertical or horizontal direction, or a size change of the transmission aperture 153b. All or at least one of the tube driver 121, the first collimator driver 131, the rotation driver 142, the carrier driver 143, the detector driver 151, and the second collimator driver 153 may be omitted in some exemplary embodiments.

The first storage unit 180 may store various information needed to control the CT scanner 100. The first storage unit 180 may exist inside or outside of the exterior housing 98 of the CT scanner 100. The first storage unit 180 may be a semiconductor memory device, or a magnetic disk memory device. The storage unit 180 may store data temporarily or non-temporarily.

The first communicator 192 may communicate data with the second communicator 211 of the workstation 200. The first communicator 192 may include at least one of a cable network device, such as a local area network (LAN) card, and a wireless network device, such as an antenna or a wireless communication chip.

The power source 193 may supply power to the respective components of the CT scanner 100. The power source 193 may be implemented by a generator provided in the CT scanner 100, or by a condenser for storing electric energy supplied from commercial electricity.

The CT scanner 100 may further include the input unit 195, such as a keyboard, a mouse, etc., and the output unit 197, such as a display, a speaker, etc. The input and output units 195 and 197 may be installed outside the exterior housing 98. The user may control to activate the CT scanner 100 or input a desired image quality through the input unit of the CT scanner 100. The user may obtain information about the selected tube voltage and tube current or may be provided an image of the object 99, through the output unit.

Referring to FIGS. 2 and 4A, the workstation 200 may receive various commands from the user and perform a corresponding process according to the received command. The workstation 200 may also provide the user with various processing results or various information, e.g., radiographic images scanned by the CT scanner 100. The workstation 200 may include the second CPU 210, the second communicator 211, an input unit 212, a second storage unit 213, and an output unit 214.

The second CPU 210 may perform operations such as calculation and processing, and generate control commands to control an operation of the CT scanner 100 and/or the workstation 200. In an exemplary embodiment, the second CPU 210 may perform functions of the first CPU 170 of the CT scanner 100. In this case, the first CPU 170 may be omitted. In another exemplary embodiment, the first CPU 170 may perform functions of the second CPU 210. The second CPU 210 may be implemented with integrated chips.

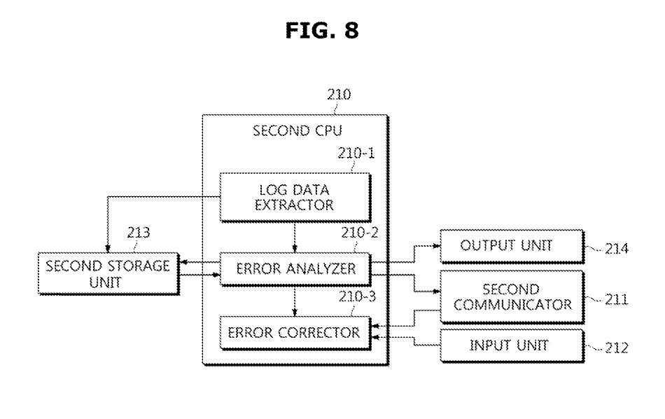

In an exemplary embodiment, the second CPU 210 may extract log data for respective components included in the CT scanner 100 and/or the workstation 200, and generate analyzed data for functional errors based on the extracted log data. For this, the second CPU 210 may include a log data extractor 210-1, an error analyzer 210-2, and an error corrector 210-3, as shown in FIG. 8.

Referring to FIG. 8, the log data extractor 210-1 may extract log data for respective components included in the CT scanner 100 and/or the workstation 200. The error detector 210-1 may detect functional errors of the radiation transmitter 110, the radiation detector 150, the second collimator 152, the tube driver 121, the first collimator driver 131, the rotation driver 142, the carrier driver 143, the detector driver 151, and the second collimator driver 153 for driving respective corresponding components, the first CPU 170, the first storage unit 180, the image processor 191, the first communicator 192, the power source 180, and the like, in addition to the carrier 95 that includes the bore 141, the gantry 140, and the cradle 97, which constitute the CT scanner 100, and extract log data for the second CPU 210, the second communicator 211, the input unit 212, the second storage unit 213, and the output unit 214, which constitute the workstation 200.

Specifically, the log data extractor 210-1 may extract log data for respective components of the CT scanner 100 and/or the workstation 200. The log data refers to data obtained by recording a history of all or part of operations of the respective components. For example, the log data may include data obtained by recording a clock speed, a current temperature, and a current load of a processor, for example, the first CPU 170 or the second CPU 210, whether a memory is currently in use, an available space of a hard disk, the memory and the hard disk, for example, the first storage unit 180 and/or the second storage unit 213, a fan speed and a temperature of a graphic unit, for example, the output unit 214, a software version, a radiation tube temperature of the gantry 140, and the like.

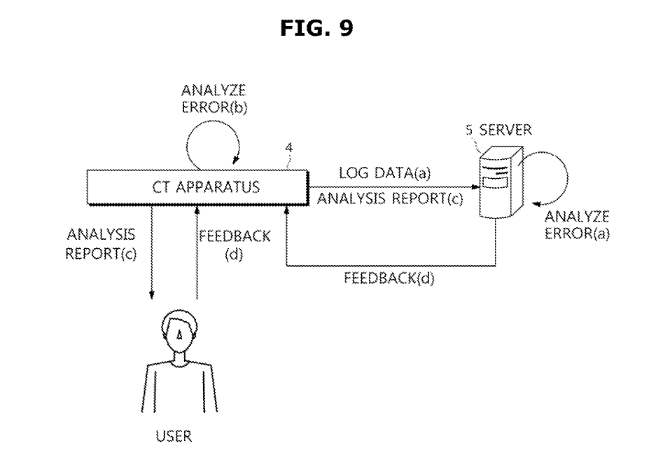

The log data extractor 210-1 may collect the log data for the respective components in real time in time series and store the log data in the second storage unit 213. Referring to FIG. 9, the log data may be sent to a server 5 owned by an external entity through the second communicator 211 in real time, thus enabling the external entity to perform error analysis (operation a).

The error analyzer 210-2 may detect and analyze functional errors of the respective components based on the extracted log data, and generate analyzed data (operations b and c). The functional errors may include mechanical errors of the respective components, input errors due to erroneous inputs by the user, and transmission errors due to transmission and/or reception of erroneous information. The mechanical errors may include read errors, memory errors, program errors, format errors, and the like. For example, the functional errors may include an abnormal state of a clock speed of the processor, a current temperature exceeding a threshold, a current load exceeding a threshold, etc. The analyzed data may be generated by classifying functional errors according to types of the respective components and/or items (or characteristics) to be checked, and stored in the second storage unit 213.

Furthermore, the analyzed data generated by the error analyzer 210-2 may be provided to the user (operation c) visually or acoustically through the output unit 14, and/or sent to the server 5 of the external entity in real time through the second communicator 211, so that the external entity may perform error correction.

The error corrector 210-3 may correct the functional errors of the respective components based on an error correction signal (or a feedback) sent by the user or the external entity (operation d). That is, the error corrector 210-3 may control an operation of the respective components to a normal operation. Specifically, if the user who receives the analyzed data through the output unit 214 inputs an error correction signal to command error correction of a component through the input unit 212, the error corrector 210-3 may correct the error of the component. In this regard, the error corrector 210-3 may correct the error of the component based on data manually input through the input unit 212 or automatically correct the error according to a program stored beforehand in the second storage unit 213.

When the error analyzer 210-2 sends the analyzed data to the server 5 of the external entity and the error corrector 210-3 receives the feedback or error correction signal from the server 5 of the external entity, the error corrector 210-3 may correct the functional errors of the respective components based on the error correction signal.

The output unit 214 may include various output means, such as display devices, speakers, light sources, or the like that may display and/or transmit information to the user.

In an exemplary embodiment, the output unit 214 may display, in graphics, the log data extracted by the log data extractor 210-1, the analyzed data generated by the error analyzer 210-2, and information regarding status of the CT scanner 100 and/or the workstation 200. The output unit 214 may be included in the workstation 200 as shown in FIG. 4A or 4C or may be included in the CT scanner 100 as shown in FIG. 4B. In an exemplary embodiment, the output unit 214 may be provided in the exterior housing 98 of the CT scanner 100.

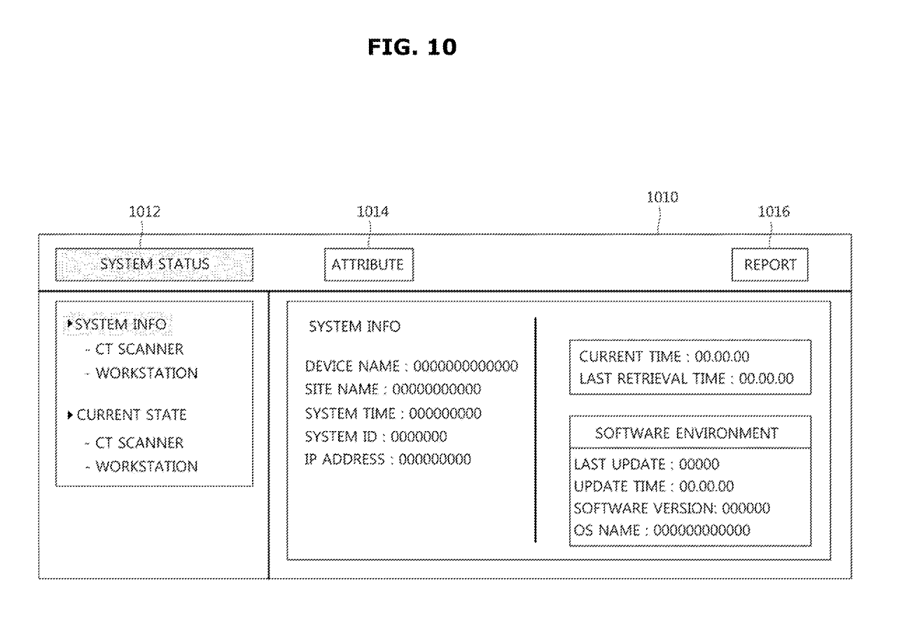

FIGS. 10, 11, 12A, 12B, 12C, and 13 illustrate user interfaces output by an output unit of a CT apparatus according to exemplary embodiments.

Referring to FIG. 10, an example screen 1010 of a user interface output by the output unit 214 may display a “system status” tab 1012, an “attribute” tab 1014, and a “report” tab 1016. When the “system status” tab 1012 is selected by an input signal received through the input unit 212, system information such as a device name or software environments, a current time, and a current state such as a last updated state, of the CT scanner 100 and/or the workstation 200, as shown in FIG. 10. When the “attribute” tab 1014 is selected by an input signal received through the input unit 212, information about hardware configuration, such as CPUs, storage units, disk drivers, keyboards, displays, etc., and information about software configuration, such as operating systems (OSs), information about software for various device drivers, etc., of the CT scanner 100 and/or the workstation 200 may be provided. When the “report” tab 1016 is selected by an input signal received through the input unit 212, current state information, such as the log data or error information of the CT scanner 100 and/or the workstation 200, and/or the analyzed data with respect to the functional errors may be sent to the server 5 of the external entity through the second communicator 211.

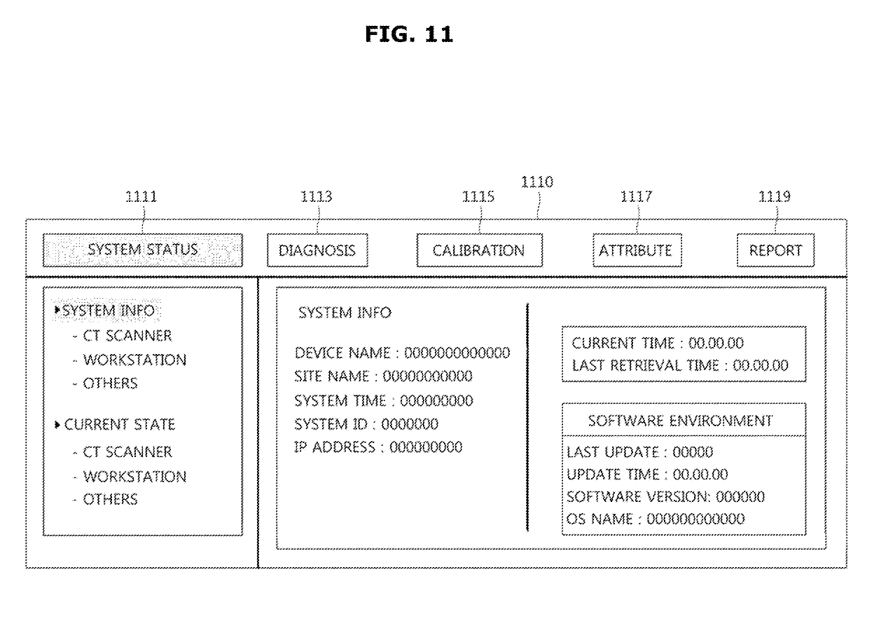

Furthermore, as shown in FIG. 11, an example screen 1110 of the user interface output by the output unit 214 may display a “system status” tab 1111, a “diagnosis” tab 1113, a “calibration” tab 1115, an “attribute” tab 1117, and a “report” tab 1119. When the “diagnosis” tab 1113 is selected by an input signal received through the input unit 212, various diagnosis information for the object obtained through the CT apparatus 4 may be provided, and when the “calibration” tab 1115 is selected, a screen for adjusting, for example, a color balance and other characteristics of the radiographic image may be provided. The system information output according to the selection of the “system status” tab 1111 is not limited to information about the CT scanner 100 and/or the workstation 200, but may include information about other devices, such as image processing devices.

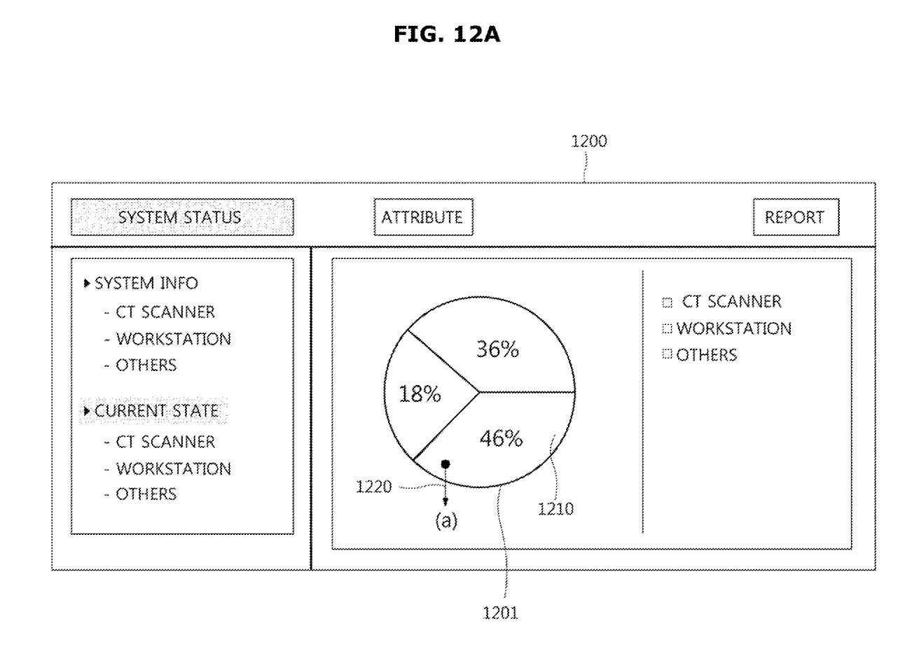

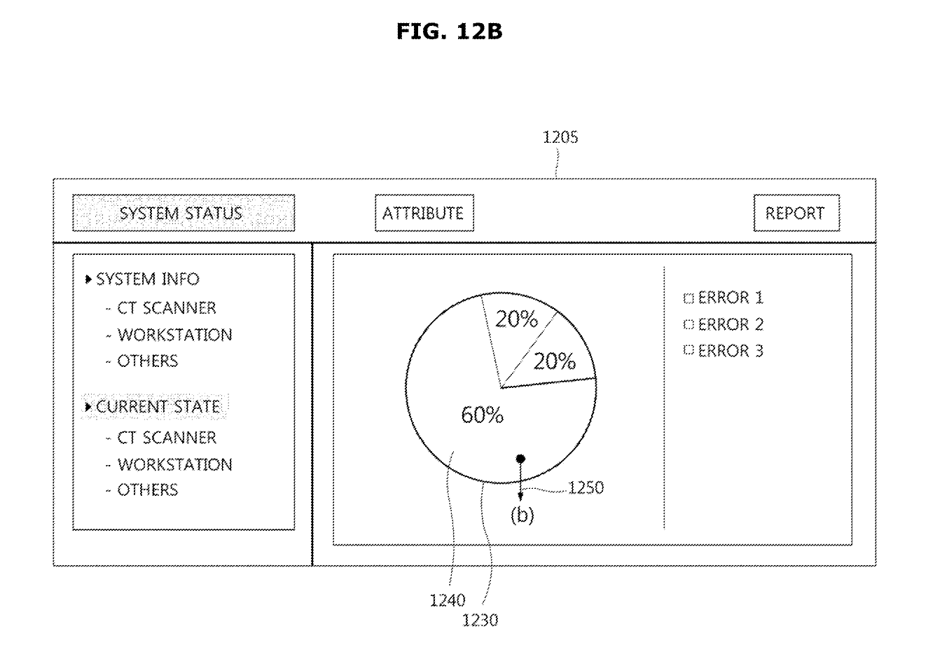

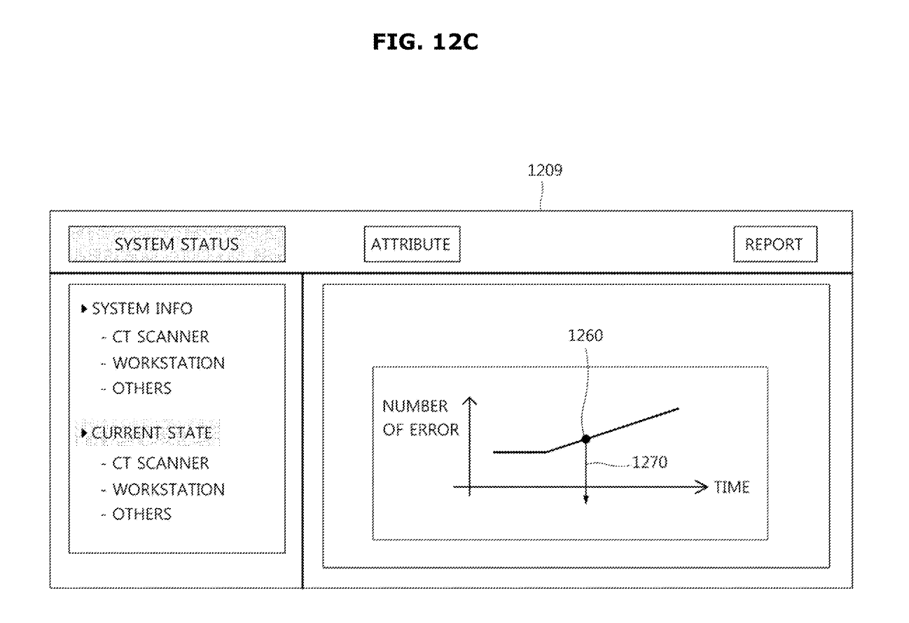

The output unit 214 may output, in graphics, the analyzed data regarding functional errors of the CT scanner 100, the workstation 200, or other devices generated by the error analyzer 210-2. The analyzed data in graphics may be output in the form of a chart as shown in FIG. 12A, or in various manners that may intuitively represent numerical data e.g., in graphs, tables, etc. For example, in an example screen 1200 of FIG. 12A, with respect to the analyzed data regarding functional errors of the CT scanner 100, the workstation 200, or other devices, the output unit 214 may display percentage information of the number of occurrences of functional errors in the respective devices using a graphical diagram 1201. For example, the graphical diagram 1201 may be divided into three regions, respectively corresponding to the CT scanner 100, the workstation 200, and other devices, and the three regions of the graphical diagram 1201 may be represented in different colors to distinguish from one another.

The output unit 214 may output the analyzed data such that a device having a greater number of occurrences of functional errors is represented by a wider area in the graphical diagram 1201. However, the method of representing the analyzed data is not limited to the example described above. For example, the output unit 214 may output the analyzed data by performing various image processing, such as increasing or decreasing brightness or representing in a particular color with respect to an area in the graphical diagram 1201 corresponding to a device having a greater number of occurrences of functional errors. In addition to outputting percentage information about the number of occurrences of functional errors of the respective devices, the output unit 214 may output the number of occurrences of functional errors according to types of the functional errors or according to time.

In an exemplary embodiment, the user may select a portion 1210 related to the CT scanner 100 of the graphical diagram 1201 as shown in FIG. 12A through the input unit 212. For example, the output unit 214 may be coupled to the input unit 212 that is implemented as a touch screen, and the user may select the portion 1210 by using a touch operation, e.g., a drag 1220, on the touch screen. If the user selects the portion 1210 related to the CT scanner 100 as shown in FIG. 12A, the output unit 214 may organize functional errors associated with the CT scanner 100 and output the functional errors associated with the CT scanner 100 according to types, e.g., an error 1, an error 2, and an error 3, as, for example, in a screen 1205 shown in FIG. 12B. For example, the error 1 may be a functional error associated with the first CPU 170 of the CT scanner 100, the error 2 with the first storage unit 180 of the CT scanner 100, and the error 3 with the input and output units 195 and 197 of the CT scanner 100. Alternatively, the error 1 may be mechanical errors, the error 2 may be transmission errors, and the error 3 may be input errors. The analyzed data may be generated based on types of the respective components and/or items to be checked of the CT scanner 100 and/or the workstation 200, as described above.

The output unit 214 may output data such that an error type (e.g., error 1, error 2, or error 3) having a greater number of occurrences of functional errors is represented by a wider area of a graphical diagram 1230. However, the method of representing the analyzed data is not limited to the example described above. For example, the output unit 214 may output the analyzed data by performing various image processing on the analyzed data, such as increasing or decreasing brightness or representing in a particular color with respect to an area of the graphical diagram 1230 corresponding to a type (e.g., error 1, error 2, or error 3) of the functional errors having a greater number of occurrences of functional errors.

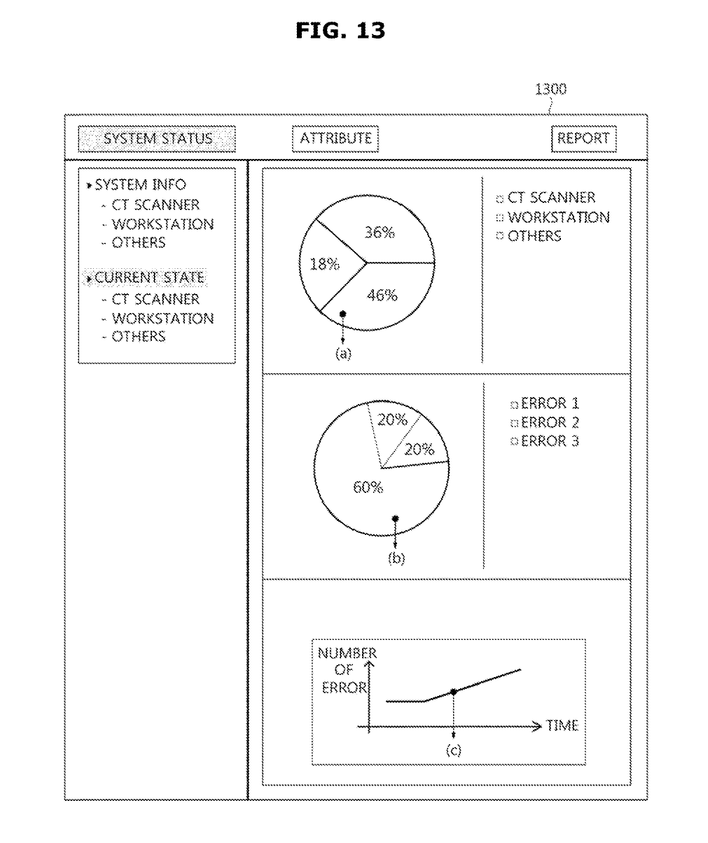

Similarly, for example, if the user selects (e.g., using a drag 1250) a portion 1240 related to the type of the error 1 from the graphical diagram 1230 through the input unit 212 as shown in FIG. 12B, the output unit 214 may display the number of occurrences of functional errors of the type of the error 1 using a graph in time series, as, for example, in a screen 1209 shown in FIG. 12C. Moreover, the output unit 214 may display the log data or the analyzed data associated with a particular point 1260 in response to an input (e.g., a drag 1270) of the user, and may display the analyzed data shown in FIGS. 12A, 12B, and 12C on a single screen 1300 as shown in FIG. 13. However, these are only examples, and the analyzed data may be output by the output unit 214 in various other forms.

Although not shown, the output unit 214 may display a tab for generating an error correction signal for correcting a particular error or errors in the analyzed data. In this case, upon reception of an error correction signal generated in response to an input on the tab for generating the error correction signal through the input unit 212, the error corrector 210-3 may control operations of the respective components of the CT scanner 100 and/or the workstation 200 based on the error correction signal.

The second communicator 211 may communicate data with the first communicator 192 of the CT scanner 100. The second communicator 211 may include a cable network device, such as a LAN card, and a wireless network device, such as an antenna or a wireless communication chip.

The input unit 212 may receive various information from the user. For example, the input unit 212 may receive a setting value for controlling quality of the radiographic image to be scanned, and inputs on various tabs of the user interface, e.g., a tab for generating an error correction signal to correct an error, etc., from the user. The input unit 212 may include various input devices, such as a keyboard, a mouse, a keypad, a trackball, a track pad, a touch pad, a touch screen, etc.

The ‘user’ may include a medical person or a hospital staff who performs diagnosis on the object, and may include a doctor, a radiographer, a nurse, etc., but is not limited thereto and may be anyone who uses the CT apparatus 4.

The second storage unit 213 may store various information provided from the second CPU 210. The second storage unit 213 may also store the log data extracted by the log data extractor 210-1 and the analyzed data generated by the error analyzer 210-2. The log data refers to data obtained by recording a history of all or portion of operations of the respective components. For example, as described above, the log data may include data obtained by recording a clock speed, a current temperature, and a current load of a processor, whether a memory is currently in use, an available space of a hard disk, the memory, a fan speed and a temperature of a graphical unit, a software version, a radiation tube temperature of the gantry 140, and the like. The analyzed data may include data related to functional errors and may be generated by the error analyzer 210-2 by classifying functional errors according to types of the respective components and/or items to be checked on the CT scanner 100 and/or the workstation 200.

The second storage unit 213 may be a semiconductor memory device and/or a magnetic disk memory device for temporarily or non-temporarily storing data.

The second storage unit 213 may largely include a program section and a data section.

The program section may store a program for controlling an operation of the CT scanner 100 and/or the workstation 200 and an OS for booting the CT scanner 100 and/or the workstation 200. The program section may include a program with respect to an operation of the first CPU 170 and/or the second CPU 210. Furthermore, the program section may include a program for outputting a user interface as shown in FIGS. 10 to 13.

The data section 622 is a section for storing data generated by using the CT scanner 100 and/or the workstation 200, and may store the log data extracted by the log data extractor 210-1 in real time and the analyzed data generated by the error analyzer 210-2.

The log data and the analyzed data stored in the second storage unit 213 may be stored in the second storage unit 213 of the workstation 200 and/or the first storage unit 180 of the CT scanner 100.

The workstation 200 may be omitted in some exemplary embodiments, and some of the components of the workstation 200 may be provided in the CT scanner 100 instead of the workstation 200, as shown in FIG. 4B.

The radiographic imaging apparatus 1 may include a DR device, a mammography device, or any other imaging device that generates radiation by applying a tube voltage and a tube current to e.g., the radiation tube and captures a radiographic image by using the radiation.

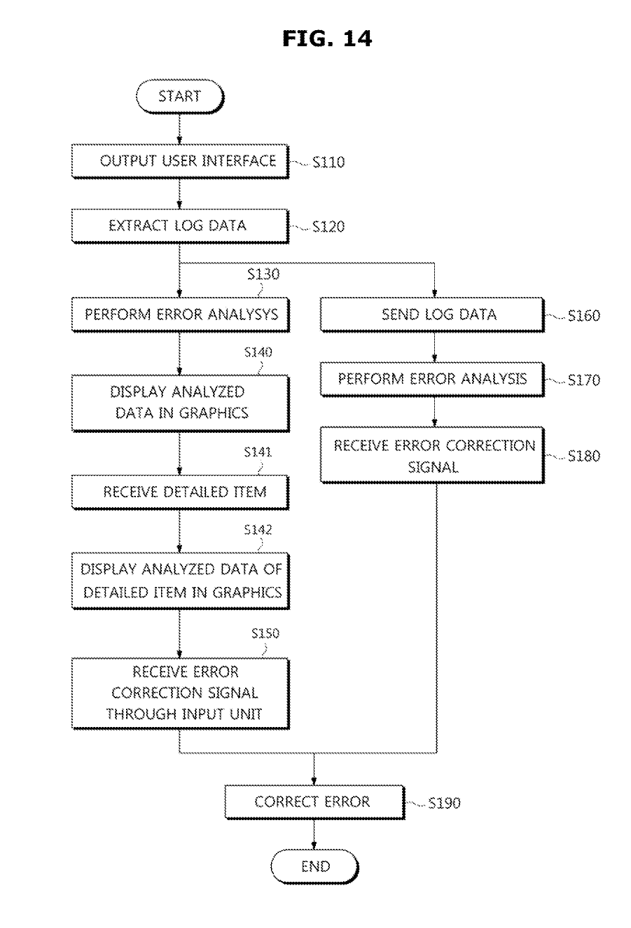

Referring to FIG. 14, when the user interface is output in operation S110, the CT apparatus 4 extracts log data for respective components of the CT scanner 100 and/or the workstation 200 in operation S120. The user interface may be stored in the first storage unit 180 and/or the second storage unit 213 and may provide various tabs to run programs and view particular data in response to a user selection, e.g., a user selection of a graphic icon associated with a program. The particular data may correspond to the analyzed data for functional errors analyzed by the CT apparatus 4. Outputting the user interface in operation S110 may be performed after performing a separate user authentication procedure, and the user authentication procedure may include a general authentication process, such as a password setting process, an email authentication process, or the like.

The log data refers to data obtained by recording a history of all or part of operations of the respective components, and is described in detail above.

The CT apparatus 4 performs error analysis in response to the user's input signal in operation S130, or sends the log data or data associated with errors of the respective components to an external server in operation S160.

In the case of performing error analysis in operation S130, the CT apparatus 4 detects and analyzes functional errors of the respective components based on the extracted log data, and outputs the resultant analyzed data to the user in graphics, in operation S140. The functional errors may include mechanical errors of the respective components, input errors due to erroneous inputs by the user, and transmission errors due to transmission and/or reception of erroneous information. The mechanical errors may include read errors, memory errors, program errors, format errors, and the like. For example, the functional errors may include an abnormal state of the clock speed of the processor, a current temperature exceeding a threshold, a current load exceeding a threshold, etc. The analyzed data may be generated in real time by classifying functional errors according to types of the respective components and/or items to be checked.

The analyzed data in graphics may be output in the form of a chart, or in various manners that may intuitively represent numerical data e.g., in graphs, tables, etc. For example, the CT apparatus 4 may output the number of occurrences of functional errors per device, per type, per hour, or the like in the form of a graph, such that a device, a type, or a point in time corresponding to a greater number of occurrences of functional errors is represented by a wider area or a higher position in the graphics. However, the method of representing the number of occurrences of functional errors is not limited to this, and the CT apparatus 4 may output the analyzed data by performing various image processing on the analyzed data, such as increasing or decreasing brightness or representing in a particular color with respect to a portion of the graphics corresponding to a device, a type, or a point in time corresponding to a greater number of occurrences of functional errors.

The CT apparatus 4 may receive a detailed item associated with a functional error from the user through the input unit, in operation S141. The detailed item may correspond to at least one of a device, a type, and a point in time of the functional error, which is designated by the user to be analyzed in detail.

The CT apparatus 4 may output, in graphics, the analyzed data that corresponds to the detailed item, e.g., the workstation 200, designated by the user, in operation S142.

The analyzed data that corresponds to the detailed item may be output in the form of a chart, or in various manners that may intuitively represent numerical data e.g., in graphs, tables, etc. For example, when the detailed item ‘workstation’ is received, the CT apparatus 4 may output the number of occurrences of functional errors of the workstation 200 per component of the workstation 200, per type, per hour, or the like in a graphical form, e.g., a graph, such that a component, a type, or a point in time corresponding to a greater number of occurrences of functional errors is represented by a wider area or a higher position in the graphical form.

However, the method of representing the analyzed data is not limited to this, and the CT apparatus 4 may output the analyzed data by performing various image processing on the analyzed data, such as increasing or decreasing brightness or representing in a particular color with respect to an area of the graphical form according to a component, a type, or a point in time corresponding to a greater number of occurrence of functional errors with respect to the detailed item, e.g., the workstation 200.

The CT apparatus 4 receives an error correction signal from the user through the input unit in operation S150, and controls the respective components of the CT apparatus 4 to correct corresponding errors in operation S190. Operations of the respective components may be controlled based on manually input instructions, or automatically correct the error according to a pre-stored program. The analyzed data may be output, not exclusively, through a separate output unit, and may be posted on the Internet or sent to the user's email through the second communicator 211.

A process of performing error analysis may be conducted by an external server in operation S170, and the external server may include e.g., a server owned by an external entity (e.g., a company). When the server performs error analysis and sends the CT apparatus 4 an error correction signal as a result of the error analysis, the CT apparatus 4 receives the error correction signal in operation S180 and controls the respective components of the CT apparatus 4 to correct the errors. Operations of the respective components may be controlled based on manually input instructions, or automatically correct the error according to a pre-stored program.

Accordingly, the method for controlling the CT apparatus 4 may be performed by the second CPU 210 of the workstation 200, as shown in FIG. 8, but is not limited thereto and may be performed by the first CPU 170 of the CT scanner 100 and/or any other device.

The CT apparatus 4 may be a radiation therapy management System (RMS)), and manage and store data in cooperation with an electronic medical record (EMR) system, an order communication system (OCS), a picture archiving and communication System (PACS), and/or a radiation treatment planning (RTP) system. The EMR system and the OCS constitute a part of a hospital information system (HIS). Configurations, functions, and operations of the EMR system, OCS, PACS, and the RTP system are well known to those ordinary skilled in the art, so the description thereof is omitted.

According to the CT apparatus 4 and the method for controlling the same according to the exemplary embodiments as described above, the user may easily obtain content of functional errors and intuitively recognize the functional errors of respective components of the CT scanner 100 and/or the workstation 200, and may command the CT apparatus 4 to perform error correction. Furthermore, the user may be provided with analyzed data in real time or send log data to an external server, thus enabling the external server to perform error analysis.

According to the radiographic imaging apparatus and the method for controlling the same according to the exemplary embodiments, functional errors of a radiographic imaging unit and/or a workstation may be analyzed in real time and the analyzed data may be observed by the user systematically and intuitively. In addition, in the radiographic imaging apparatus and the method for controlling the same according to the exemplary embodiments, the user may be provided with the analyzed data in graphics in real time, and may thus immediately recognize the functional errors of the radiographic imaging unit and/or the workstation.

The foregoing exemplary embodiments and advantages are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A radiographic imaging apparatus comprising:
a radiation scanner; and
a workstation configured to control the radiation scanner, output analyzed data for a functional error of at least one of the radiation scanner and the workstation in graphics, and output analyzed data for an item of the functional error in response to an input of a user.

2. The radiographic imaging apparatus of claim 1, wherein the workstation is configured to output the analyzed data for the item of the functional error in graphics.

3. The radiographic imaging apparatus of claim 1, wherein the workstation includes a storage unit in which the analyzed data for the functional error are classified and stored, and
the workstation is configured to generate the analyzed data for the functional error based on at least one of a type of a component and a characteristic of the at least one of the radiation scanner and the workstation, and store the analyzed data for the functional error in the storage unit.

4. The radiographic imaging apparatus of claim 1, wherein the workstation is configured to output the analyzed data for the functional error as at least one of a chart and a graph.

5. The radiographic imaging apparatus of claim 1, wherein the workstation is configured to output the analyzed data for the functional error in time series.

6. The radiographic imaging apparatus of claim 1, wherein the workstation is configured to detect the functional error in real time.

7. The radiographic imaging apparatus of claim 1, wherein the workstation is configured to transmit data regarding an operation of the at least one of the radiation scanner and the workstation to a server, and control the operation of the at least one of the radiation scanner and the workstation based on an error correction signal that is received from the user or the server.

8. The radiographic imaging apparatus of claim 1, wherein the workstation is configured to control an operation of the at least one of the radiation scanner and the workstation based on an error correction signal that is received from the user.

9. A radiographic imaging apparatus comprising:
a controller configured to detect a functional error of a radiation scanner and generate analyzed data for the functional error;
an output unit configured to output the analyzed data for the functional error in graphics; and
an input unit configured to receive an input of an item of the functional error,
wherein the controller is configured to generate analyzed data for the item of the functional error in response to receiving the input of the item of the functional error, and control the output unit to output the analyzed data for the received item.

10. The radiographic imaging apparatus of claim 9, wherein the input unit is configured to receive an error correction signal from a user and the controller is configured to control an operation of the radiation scanner based on the error correction signal.

11. The radiographic imaging apparatus of claim 9, further comprising:
a storage unit in which the analyzed data for the functional error are classified and stored,
wherein the controller is configured to generate the analyzed data for the functional error based on at least one of a type of a component and a characteristic of the radiation scanner, and store the analyzed data for the functional error in the storage unit.

12. The radiographic imaging apparatus of claim 9, wherein the controller is configured to control the output unit to output the analyzed data for the functional error as at least one of a chart and a graph.

13. The radiographic imaging apparatus of claim 9, wherein the output unit is configured to output the analyzed data for the functional error in time series.

14. The radiographic imaging apparatus of claim 9, wherein the controller is configured to detect the functional error in real time.

15. The radiographic imaging apparatus of claim 9, wherein the analyzed data for the received item of the functional error includes analyzed data for at least one of a mechanical error of the radiation scanner, an input error due to an erroneous input of a user, and a transmission error due to transmission or reception of erroneous information.

16. A method for controlling a radiographic imaging apparatus, the method comprising:
generating analyzed data for a functional error of at least one of a radiation scanner and a workstation configured to control the radiation scanner;
outputting the analyzed data for the functional error in graphics;
receiving an item of the functional error;
generating analyzed data for the item of the functional error; and
outputting the analyzed data for the item of the functional error.

17. The method of claim 16, further comprising:
storing the analyzed data for the functional error in a storage unit.

18. The method of claim 16, wherein the generating the analyzed data for the functional error comprises:
generating the analyzed data for the functional error based on at least one of a type of a component and a characteristic of the at least one of the radiation scanner and the workstation.

19. The method of claim 16, wherein the outputting the analyzed data for the functional error comprises:
outputting the analyzed data for the functional error as at least one of a chart and a graph.

20. The method of claim 16, wherein the outputting the analyzed data for the functional error comprises:
outputting the analyzed data for the functional error in time series.

21. The method of claim 16, further comprising:
receiving an error correction signal from a user; and
controlling an operation of the at least one of the radiation scanner and the workstation based on the error correction signal.

22. The radiographic imaging apparatus of claim 1, wherein the workstation is configured to generate analyzed data for respective functional errors of the radiation scanner and the workstation, and output information about numbers of occurrences of the respective functional errors of the radiation scanner and the workstation in graphics.

23. The radiographic imaging apparatus of claim 22, wherein the item of the functional error comprises at least one of a device, a type, and a point in time of the functional error.

24. The radiographic imaging apparatus of claim 23, wherein, in response to an input of selecting one of the radiation scanner and the workstation, the workstation is configured to generate the analyzed data for a type of the functional error of the selected one of the radiation scanner and the workstation.
